(19) United States
(12) Patent Application Publication
(10) Pub. No.: US 2014/0107739 A1
(43) Pub. Date: Apr. 17, 2014

(54) REUSABLE HOT / COLD WRAP

(71) Applicant: Christopher Kirkman, San Diego, CA (US)

(72) Inventor: Christopher Kirkman, San Diego, CA (US)

(21) Appl. No.: 13/829,226

(22) Filed: Mar. 14, 2013

Related U.S. Application Data
(63) Continuation-in-part of application No. 13/553,507, filed on Jul. 19, 2012.

(51) Int. Cl. A61F 7/02

(52) U.S. Cl. CPC A61F 7/02
USPC 607/112; 156/66

(57) ABSTRACT

A reusable wrap for holding and securing a substantially planar heat transfer pouch to a body part includes a substantially planar container made of a flexible non-woven material and formed as a rectangular pocket. The pocket includes an open end that is sized to receive the planar heat transfer pouch. The planar heat transfer pouch can be, for example, a flexible rectangular shaped pouch having a plurality of coplanar pouch pockets for holding ice lumps. A thermal barrier can be positioned in the container pocket and located to interpose the heat transfer pouch between the thermal barrier and the body part. An elongated strap is attached to the container and includes at least one fastener to secure the container and heat transfer pouch against the surface of the body part when the strap is wrapped around the body part.

This application is a continuation-in-part of application Ser. No. 13/553,507, filed Jul. 19, 2012, to inventor Christopher Kirkman, (attorney docket number 11607.2) which is currently pending. The entire contents of application Ser. No. 13/553,507 are incorporated herein by reference.

### FIELD OF THE INVENTION

The present invention pertains generally to ice wraps. More particularly, the present invention pertains to reusable flexible ice wraps that can be filled and frozen by the user. The present invention is particularly, but not exclusively, useful as an ice wrap having an integral strap for attaching a flexible, frozen portion of the ice wrap to a user's body.

### BACKGROUND OF THE INVENTION

The application of ice or another cold substance to a body part is often prescribed after an injury to reduce pain and/or swelling. In addition, cold therapy can be useful to reduce pain and/or swelling in a previously injured joint or muscle, for example, after an activity such as working or exercise.

To be effective, it can be important that the cold applicator conform to the particular anatomical region requiring therapy. In this regard, the anatomical regions that often require therapy vary considerably in shape and size. These can include, for example, the knee, ankle, shoulder, elbow and wrist. In addition to the anatomical variation within a particular individual, the size and shape of different individuals varies greatly across the general population. For this reason, it is often desirable to have a cold applicator that is flexible to fit most if not all of the different anatomical shapes and sizes.

Once applied, it is generally desirable to leave the cold wrap in place for an extended period of time. Moreover, the therapeutic value of a cold application can often be increased if the application is accompanied by a moderate amount of compression. In this regard, it is often desirable to affix the cold applicator to the affected body part to relieve the person from the chore of holding the cold applicator in place. For this purpose, various attachment methods have been suggested. In most cases, it is desirable that the cold applicator be able to be applied and affixed by the person requiring treatment, and some instances can be accomplished with only the use of one hand. Further, it is desirable that the cold applicator be reusable.

After the applicator has been applied to the affected body part, the temperature of the applicator medium, whether hot or cold, will begin to change, reducing the therapeutic value of the application. When ice is used as the applicator medium, this temperature change can result in ice melting producing water. The water is typically undesirable as it may drip all over the user's clothing and leave the user generally wet and uncomfortable.

In light of the above, it is an object of the present invention to provide a reusable, flexible ice applicator that is conformable to several different parts of the body. It is another object of the present invention is to provide an ice applicator system that can be easily affixed to the user. It is still another object of the present invention is to provide a hot/cold wrap that resists changes in the applicator medium's temperature during the course of a treatment. Yet another object of the present invention is to provide a reusable hot/cold wrap and corresponding methods of use which are easy to use, relatively simple to implement, and comparatively cost effective.

### SUMMARY OF THE INVENTION

In accordance with the present invention, a device for transferring heat from a body part includes a segmented pouch. To create the pouch, two flexible plastic layers (i.e. an upper and lower layer) are heat bonded together at their peripheries. In addition, a fluid port is established for the pouch to allow a fluid such as water to be introduced into the pouch. Within the pouch, a plurality of substantially coplanar pockets for holding a respective plurality of frozen fluid lumps are formed by heat bonding the layers together in a preselected pattern. With this arrangement, the pouch and frozen fluid lumps establish an ice wrap that is sufficiently flexible to conform to a surface of the body such as the knee, ankle, wrist, etc.

For the device of the present invention, an elongated strap is attached to the pouch to secure the pouch in place once the pouch and frozen fluid lumps have been applied to the surface of the body. Structurally, for the present invention, the strap can be made of nonwoven fabric and it is sized to be long enough to wrap around the pouch and a targeted body part.

In one embodiment, a first end of the strap is attached to the pouch and a piece of hook fastener is affixed to the strap adjacent the first end. With this arrangement, the hook fastener and nonwoven fabric establish a fastening system when the strap is folded over and across the pouch to maintain the pouch against the surface of the body part.

In another embodiment, a first end of the strap is attached to the pouch and a piece of hook fastener is affixed to the strap adjacent a second end of the strap. With this arrangement, the hook fastener attaches to the nonwoven fabric after the strap has been fully wrapped around the pouch and body to hold the pouch in place against the body and provide compression.

Alternatively, a first piece of hook fastener can be affixed to the strap adjacent to the first end of the strap (on a first side of the strap) and a second piece of hook fastener can be affixed to the strap adjacent the second end of the strap (on a second side of the strap). With this arrangement, the pouch can be applied to a targeted body part and the strap wrapped around the body part and over the pouch. A first fastening attachment is then made between the strap and the first piece of hook fastener once the strap covers the pouch. The remainder of the strap is then wrapped around the body part and a second fastening attachment is made between the strap and the second piece of hook fastener to secure the strap in place.

For the present invention, the fluid port for introducing fluid into the pouch can include a tapered input channel that is formed at a peripheral section of the pouch by heat bonding the upper and lower layers together in a preselected pattern. In one embodiment, a one-way fluid valve such as a duck-bill valve may be inserted into the input channel and attached to the upper and lower layers. Alternatively, a sliderless plastic zipper may be formed at a peripheral section of the pouch.

To establish the plurality of coplanar pockets for holding a respective plurality of frozen fluid lumps, the upper layer and the lower layer are connected in a pattern that is formed by heat bonding the layers together. The pattern can include a first plurality of partitions with each partition in the first plurality formed with fluid channels that interconnect adjacent pockets. For the pattern, each partition in the first plurality is aligned substantially parallel to other partitions in the first plurality. Also, the pattern includes a second plurality of partitions with each partition in the second plurality formed with fluid channels that interconnect adjacent pockets. For the pattern, each partition in the second plurality is aligned substantially parallel to other partitions in the second plurality and substantially perpendicular to the partitions of the first plurality.

In another aspect of the present invention, a reusable hot/cold wrap for holding and securing a substantially planar heat transfer pouch to a body part includes a substantially planar container. For this aspect, the planar container can be made of a stretchable, non-woven material and formed as a rectangular pocket. The pocket is formed from the stretchable material having an open end that is sized to receive the planar heat transfer pouch. To form the pocket, a piece of non-woven material can be folded over onto itself to establish an upper layer that overlays an equally sized lower layer. After folding, a section of the upper layer's peripheral and a corresponding section of the lower layer's peripheral are permanently joined together, for example, by heat bonding to establish the pocket. For this aspect, the planar heat transfer pouch can be, for example, a flexible, rectangular shaped pouch, as described above, having a plurality of coplanar pouch pockets holding a medium, such as an ice lump. Alternatively, the planar heat transfer pouch can hold hot water, cold water or any other suitable heat transfer medium known in the pertinent art.

For the reusable hot/cold wrap, a thermal barrier can be located in the container pocket and positioned to interpose the heat transfer pouch between the thermal barrier and the body part. With this arrangement, the heat transfer medium, e.g. ice, will maintain its temperature longer. For example, the thermal barrier can be made of flexible, insulating material such as a sheet of closed cell plastic foam.

In addition to the thermal barrier, the reusable wrap can include an elongated strap that is made of stretchable material such as a non-woven fabric. For this aspect, the strap is formed having a first end and a second end, and is attached to the container with the first end of the strap affixed to the container near the opening of the pocket. For example, sewing or heat bonding can be used to affix the strap to the container. Alternatively, in some instances, the strap and portions or all of the container can be made of a single piece of nonwoven material.

A first fastener is attached to the strap at the second strap end to secure the container against the surface of the body part when the strap is wrapped around the body part. A second fastener can be attached to the strap at the first strap end, with both fasteners located on the same side on the strap. For example, the fasteners can include a plurality of hook-like structures that are engageable with non-woven material. With this arrangement, the fasteners can engage the container material and/or the strap material.

To use or reuse the wrap, the heat transfer pouch is prepared by filling with water and freezing. Then the heat transfer pouch is inserted into the container pocket. In some cases, the heat transfer pouch can be filled and conditioned to a desired temperature, e.g. frozen, while the heat transfer pouch is in the pocket of the wrap. With the wrap prepared, the container holding the heat transfer pouch is positioned against the body part and the strap is wrapped around the body part. Depending on the size of the body part, the fasteners engage with the non-woven fabric of the container and/or strap to secure the container holding the heat transfer pouch firmly in place.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a segmented ice wrap device in accordance with the present invention shown after applying the pouch portion of the device on a person's knee;

FIG. 2 is a plan view of the segmented ice wrap device shown in FIG. 1;

FIG. 2A is a cross sectional view of the pouch portion of the segmented ice wrap shown in FIG. 2 as seen along line 2A-2A in FIG. 2;

FIG. 3 is a detail view of a portion of a device enclosed by line 3-3 in FIG. 2 illustrating a first pattern of heat bond dots for creating a plurality of coplanar pockets for holding a respective plurality of frozen fluid lumps;

FIG. 4 is a detail view of a portion of a device enclosed by line 3-3 in FIG. 2 illustrating another pattern of heat bond dots for creating a plurality of coplanar pockets for holding a respective plurality of frozen fluid lumps;

FIG. 5 shows another embodiment of a device in accordance with the present invention having a tapered fluid port; and

FIG. 6 shows another embodiment of a device in accordance with the present invention having a fluid port formed with a constriction;

FIG. 7 shows an embodiment of a reusable hot/cold wrap in accordance with the present invention; and

FIG. 8 is a cross-sectional view of a reusable hot/cold wrap as seen along line 8-8 in FIG. 7, shown with a heat transfer pouch inserted in the pocket of the reusable hot/cold wrap.

### DESCRIPTION OF THE PREFERRED EMBODIMENTS

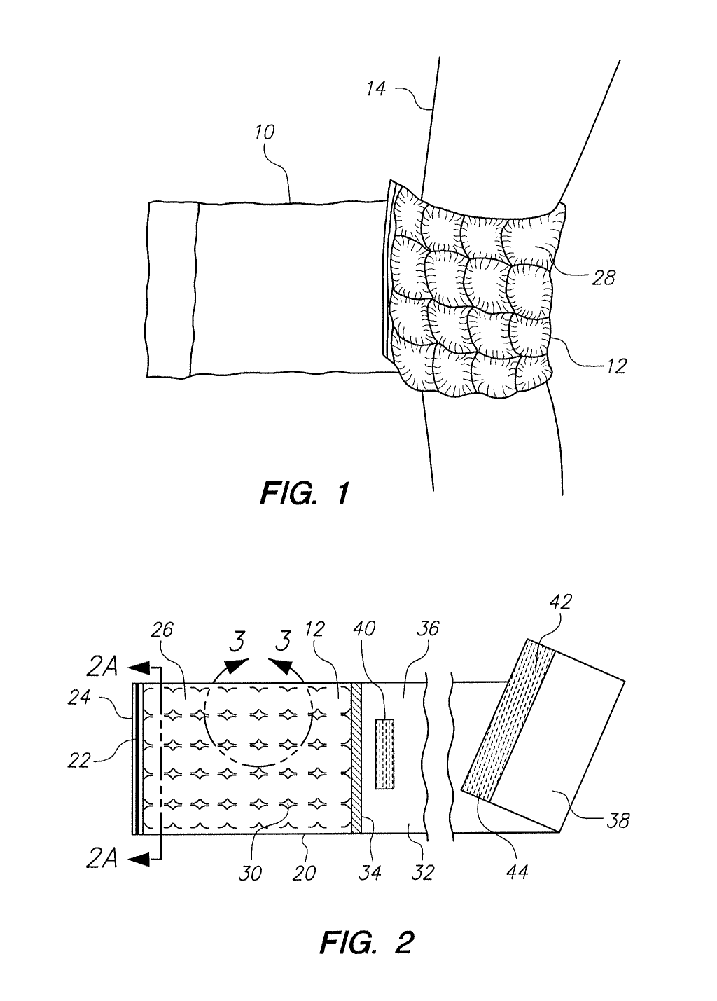

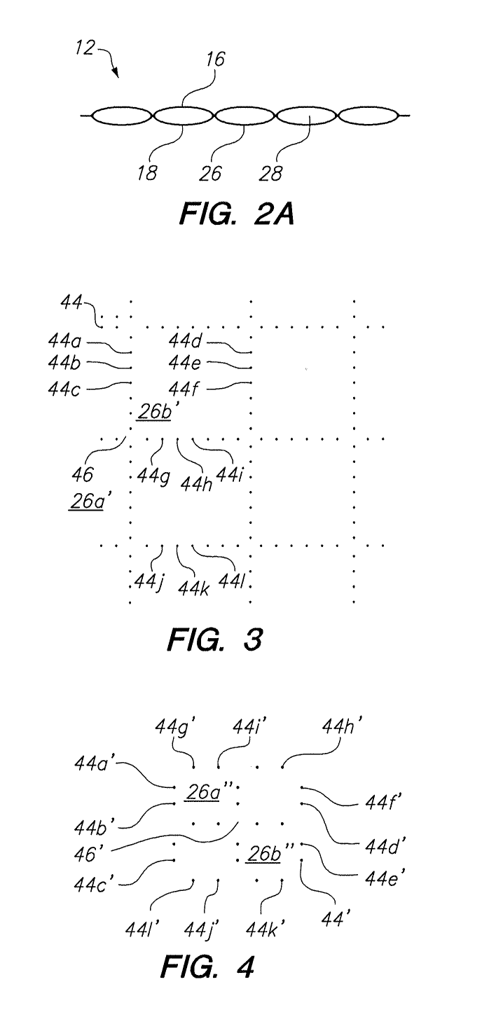

Referring initially to FIG. 1, an ice wrap device 10 having a segmented pouch 12 in accordance with the present invention is shown. Specifically, FIG. 1 shows the device 10 after the pouch 12 has been applied to a surface of a person's leg 14 to cover their knee. As best appreciated by cross referencing FIGS. 1, 2 and 2A, the pouch 12 may be generally rectangular shaped and may be formed from an upper layer 16 and a lower layer 18 of plastic that are heat bonded together at a peripheral section 20 to create the pouch 12.

FIG. 2 shows that a fluid port 22 can be established for the pouch 12 at a peripheral section 24 to allow a fluid such as water or gel to be introduced into the pouch 12. For the embodiment shown in FIG. 2, the fluid port 22 can be a sliderless plastic zipper sometimes referred to generically as a so-called Ziploc closure.

Continuing with cross-reference to FIGS. 1, 2 and 2A, it can be seen that within the pouch 12, a plurality of substantially coplanar pockets 26 for holding a respective plurality of frozen fluid lumps 28 are formed by heat bonding the layers 16, 18 together in a preselected pattern 30. With this arrangement, the pouch 12 and frozen fluid lumps 28 establish an ice wrap device 10 that is sufficiently flexible to conform to a surface of the body such as the knee, ankle, wrist, etc. For example, the plastic sheets used for the upper layer 16 and lower layer 18 may be made of a clear and/or transparent material that is sufficiently stretchable to accommodate a freezing of the fluid into lumps 28 within the respective pockets 26. For example, the sheet material may be a combination of nylon and polyethylene having a thickness of less than approximately four mils.

FIGS. 1 and 2 also show that the device 10 includes an elongated strap 32 having an end 34 that is attached to the pouch 12. For example, the pouch and strap 32 may be attached together using heat bonding or adhesive bonding. For the device 10, the strap 32 can be made of nonwoven fabric and is typically sized to be long enough to wrap around the pouch and a targeted body part such as the knee, ankle, elbow, etc. For example, the nonwoven fabric may be made of polypropylene and can include a texture. With this arrangement, the strap 32 can be used to secure the pouch 12 in place once the pouch 12 and frozen fluid lumps 28 have been applied to the surface of the body (see FIG. 1).

As best seen in FIG. 2, the strap 32 includes a side 36 and an opposed side 38. FIG. 2 also shows that a piece of hook fastener 40 can be affixed to the nonwoven fabric of the strap 32 on the side 36 of the strap 32 and adjacent the end 34 of the strap 32. Continuing with FIG. 2, it can be seen that another piece of hook fastener 42 can be affixed to the nonwoven fabric of the strap 32 on the side 38 of the strap 32 and adjacent an end 34 (opposite end 34) of the strap 32. For example, the hook fastener 40, 42 can be adhesively bonded and/or sewn on to the nonwoven fabric of the strap 32.

With this arrangement shown in FIGS. 1 and 2, the pouch 12 can be applied to a targeted body part and the strap 32 wrapped around the body part and over the pouch 12. A first fastening attachment is then made between the nonwoven fabric of the strap 32 and the piece of hook fastener 40 once the strap 32 covers the pouch 12. The remainder of the strap 32 is then wrapped around the body part and a second fastening attachment is made between the nonwoven fabric of the strap 32 and the piece of hook fastener 42 to secure the strap 32 in place.

FIGS. 3 and 4 show respective heat bond patterns 30′, 30″ for establish the plurality of coplanar pockets 26′; 26″ for holding a respective plurality of frozen fluid lumps (see FIG. 2A). The patterns 30′, 30″ shown can be repeated throughout the pouch 12 as shown in FIG. 2. Pattern 30′ shown in FIG. 3 is designed to produce relatively large ice lumps that are slightly rectangular and have a large side dimension of about one inch (25.4 mm). On the other hand, pattern 30″, shown in FIG. 4, is designed to produce relatively small ice lumps that are substantially square and have a side dimension of about one half inch (12.7 mm).

As shown in FIG. 3, the layers 16, 18 (see FIG. 2A) are connected in a pattern of heat bond dots 44 that are formed by applying heat to the layers 16, 18 at the location of each dot 44. Also shown in FIG. 3, the pattern 30′ can include a plurality of dots 44a-c that are arranged linearly along a first line that establishes a first partition. It can also be seen that the pattern 30′ can include a plurality of dots 44d-f that are arranged linearly along a second line that establishes a second partition. Thus, the pattern 30′ establishes a first plurality of partitions in which each partition in the first plurality is aligned substantially parallel to other partitions in the first plurality.

Also shown in FIG. 3, the pattern 30′ can include a plurality of dots 44g-i that are arranged linearly along a third line that establishes a third partition. It can also be seen that the pattern 30′ can include a plurality of dots 44j-l that are arranged linearly along a fourth line that establishes a fourth partition. Thus, pattern 30′ establishes a second plurality of partitions in which each partition in the second plurality is aligned substantially parallel to other partitions in the second plurality. In addition, as shown, for the pattern 30′, each partition in the second plurality is aligned to be substantially perpendicular to the partitions of the first plurality. With this pattern 30′ of dots 44, as illustrated by FIG. 3, a plurality of fluid channels 46 are established that interconnect adjacent pockets 26a′, 26b′.

As shown in FIG. 4, the layers 16, 18 (see FIG. 2A) are connected in a pattern of heat bond dots 44′ that are formed by applying heat to the layers 16, 18 at the location of each dot 44′. Also shown in FIG. 4, the pattern 30″ can include a plurality of dots 44a′-c′ that are arranged linearly along a first line that establishes a first partition. It can also be seen that the pattern 30″ can include a plurality of dots 44d′-f′ that are arranged linearly along a second line that establishes a second partition. Thus, the pattern 30″ establishes a first plurality of partitions in which each partition in the first plurality is aligned substantially parallel to other partitions in the first plurality.

Also shown in FIG. 4, the pattern 30″ can include a plurality of dots 44g′-i′ that are arranged linearly along a third line that establishes a third partition. It can also be seen that the pattern 30″ can include a plurality of dots 44j′-l′ that are arranged linearly along a fourth line that establishes a fourth partition. Thus, pattern 30″ establishes a second plurality of partitions in which each partition in the second plurality is aligned substantially parallel to other partitions in the second plurality. In addition, as shown, for the pattern 30″, each partition in the second plurality is aligned to be substantially perpendicular to the partitions of the first plurality. With this pattern 30″ of dots 44′, as illustrated by FIG. 4, a plurality of fluid channels 46′ are established that interconnect adjacent pockets 26a″, 26b″. Although FIGS. 3 and 4 show two exemplary patterns, it is to be appreciated that the number of dots per pocket can be varied, that linear heat bond segments may be used in place of a linear array of dots, that the pockets may be oval, square, rectangular or some other shape, and that the partitions as defined above may be non-perpendicular.

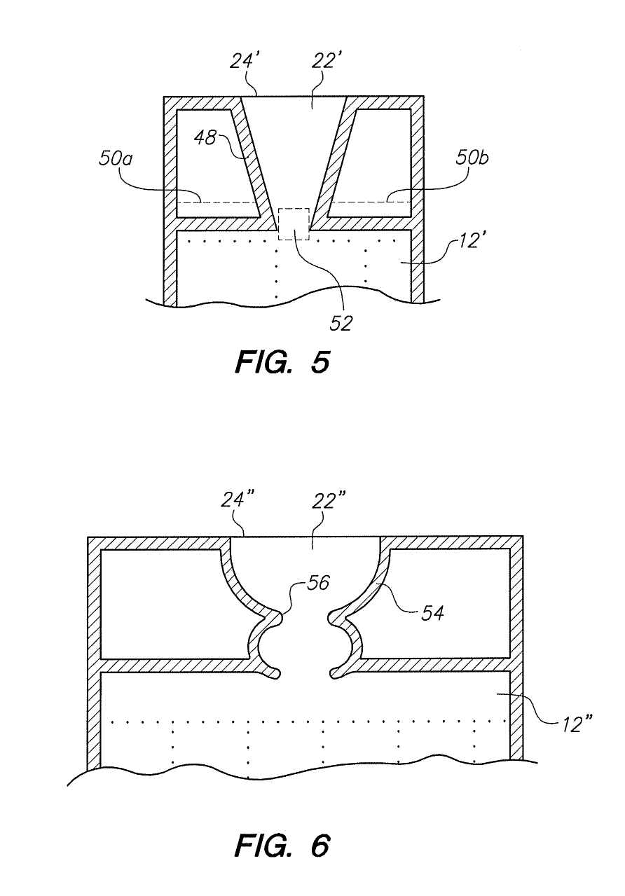

FIG. 5 shows an example of a fluid port 22′ for use in the device 10 to introduce a fluid such as water into the pouch 12′. As shown, the fluid port 22′ can include a tapered input channel 48 that is formed at a peripheral section 24′ of the pouch 12′ by heat bonding layers 16, 18 (see FIG. 2A) together in the pattern shown (heat bond shown in crossed hatching). Perforations 50a,b on each side on the input channel 48 can be provided allowing a user to tear the perforations 50a,b to establish tabs which can then the tied to close the fluid port 22′ after filling. An optional, a one-way fluid valve 52 such as a duck-bill valve may be inserted into the input channel 48 and attached to the upper and lower layers 16, 18 (see FIG. 2A), as shown.

FIG. 6 shows another example of a fluid port 22″ for use in the device 10 to introduce a fluid such as water into the pouch 12″. As shown, the fluid port 22″ can include an input channel 54 that is formed at a peripheral section 24″ of the pouch 12″ by heat bonding layers 16, 18 (see FIG. 2A) together in the pattern shown (heat bond shown in crossed hatching). As shown, input channel 54 is formed with constriction 56 which provides a venture effect to cause the input channel 54 to close at the constriction 56 in response to a flow of fluid out of the input channel 54 from the pouch 12″.

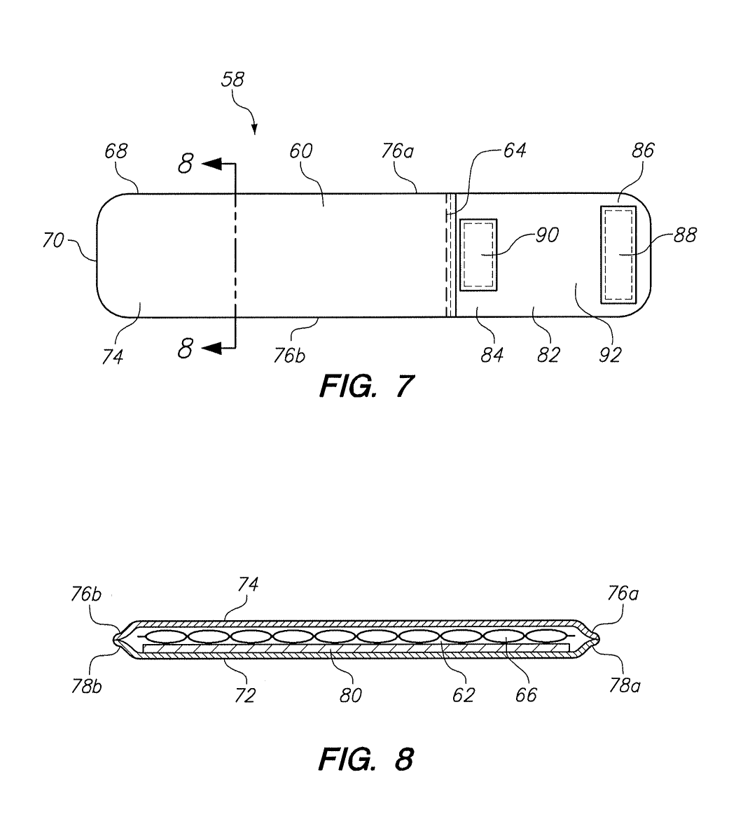

FIGS. 7 and 8 show a reusable hot/cold wrap 58 having a substantially planar container 60. For this wrap 58, the planar container 60 can be made of a stretchable, non-woven material. As best seen by comparing FIG. 7 to FIG. 8, the container 60 can be formed as a rectangular pocket 62 having an open end 64 that is sized to receive a planar heat transfer pouch 66 and a closed peripheral section 68. To form the pocket 62, a piece of non-woven material can be folded over onto itself along an edge 70 to establish a layer 72 that overlays an equally sized layer 74. After folding, peripheral sections 76a,b of the layer 72 and corresponding peripheral sections 78a,b of the layer 74 can be permanently joined together, for example, by heat bonding and/or sewing. Alternatively, in a non-folded embodiment, (not shown) two separate pieces of non-woven material can be permanently joined together along a portion of their respective edges to produce a pocket.

For wrap 58, the planar heat transfer pouch 66 can be, for example, a flexible, rectangular shaped pouch 66, as described above with reference to FIGS. 1, 2 and 2A, having a plurality of coplanar pouch pockets 26 holding a medium, such as an ice lump 28. Alternatively, for use with the wrap 58 shown in FIG. 7, the planar heat transfer pouch can hold hot water, cold water or any other suitable heat transfer medium known in the pertinent art, (not shown) and can be formed with or without individual compartments (i.e. pouch pockets).

FIG. 8 further shows that a thermal barrier 80 can be located in the pocket 62 and positioned to interpose the heat transfer pouch 66 between the thermal barrier 80 and the layer 74. The layer 74, in turn, is applied against a body part (not shown) during use. Thus, in use, the heat transfer pouch 66 is positioned between the thermal barrier 80 and the body part (not shown) in use. With this arrangement, the heat transfer medium, e.g. ice, will maintain its temperature longer. For example, the thermal barrier 80 can be made of flexible, insulating material such as a sheet of closed cell plastic foam.

Continuing with FIG. 7, it can be seen that the reusable wrap 58 can include an elongated strap 82 that is made of stretchable material such as a non-woven fabric. As shown, the strap 82 is formed having a first end 84 and a second end 86, and is attached to the container 60 with the first end 84 of the strap 82 affixed to the container 60 near the open end 64 of the pocket 62. For example, the first end 84 of the strap 82 can be sewn and/or heat bonded to the container 60. In an alternative embodiment (not shown), a strap and portions or all of the container can be made of a single piece of nonwoven material.

FIG. 7 further shows that a fastener 88 is attached to the strap 82 at the second end 86 of the strap 82 and a fastener 90 is attached to the strap 82 at the first end 84 of the strap 82. As shown, fasteners 88, 90 are located on the upper side 92 of the strap 82. In one embodiment, the fasteners 88, 90 include a plurality of hook-like structures that are releasable engageable with non-woven material. With this arrangement, the hook-like structures of the fasteners 88, 90 can engage the container material and/or the strap material when the hook-like structures are pressed onto the container material and/or the strap material with moderate pressure. Also, once applied, the hook-like structures of the fasteners 88, 90 can release/disengage from the container material and/or the strap material when the hook-like structures are pulled from the container material and/or the strap material with moderate pulling force.

Continuing with FIGS. 7 and 8, to use or reuse the wrap 58, the heat transfer pouch 66 is prepared by filling with a heat transfer medium such as water and conditioning the heat transfer medium to a desired temperature or state, e.g. freezing. Then the heat transfer pouch 66 is inserted into the container pocket 62 as shown in FIG. 8. With the wrap 58 prepared, the container 60 holding the heat transfer pouch 66 is positioned against the body part, such as the leg 14 shown in FIG. 1, and the strap 82 is wrapped around the body part. Depending on the size of the body part, the fasteners 88, 90 engage with the non-woven fabric of the container 60 and/or strap 82 to secure the container 60 holding the heat transfer pouch 66 firmly in place.

While the particular reusable hot/cold wrap and corresponding methods of manufacture as herein shown and disclosed in detail are fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A reusable wrap for holding and securing a substantially planar heat transfer pouch to a body part, the wrap comprising:
a substantially planar container forming a pocket having an open end sized to receive the planar heat transfer pouch, the container made of a flexible material to conform the container against a surface of the body part;
an elongated strap made of a flexible material having a first end and a second end, wherein the first end of the strap is affixed to the container;
a thermal barrier positioned in the pocket to interpose the heat transfer pouch between the thermal barrier and the body part; and
a fastener attached to the strap at the second strap end and engageable with the container material to secure the container against the surface of the body part when the strap is wrapped around the body part.

2. A wrap as recited in claim 1 wherein the fastener is engageable with the strap material to secure the container against the surface of the body part when the strap is wrapped around the container.

3. A wrap as recited in claim 1 wherein the container comprises an upper layer having a periphery and a lower layer having a periphery, wherein a portion of the upper and lower peripheries are permanently joined together to establish the pocket.

4. A wrap as recited in claim 3 wherein the container is rectangular.

5. A wrap as recited in claim 1 wherein the strap is stretchable and is made of a nonwoven material.

6. A wrap as recited in claim 1 wherein the container is stretchable and is made of a nonwoven material.

7. A wrap as recited in claim 6 wherein the nonwoven material is polypropylene.

8. A wrap as recited in claim 1 wherein the fastener is a first fastener and the device further comprises a second fastener attached to the strap at the first end of the strap and engageable with the container material to secure the container against the surface of the body part when the strap is wrapped around the body part.

9. A wrap as recited in claim 8 wherein the first fastener and the second fastener respectively include a plurality of hook-like structures, and wherein the hook-like structures establish a secure engagement with the container material when the fastener is placed in contact with the container material.

10. A wrap as recited in claim 1 wherein the thermal barrier is flexible and made of a closed cell plastic foam material.

11. A device for transferring heat from a body part which comprises:
a substantially planar heat transfer pouch;
a substantially planar container forming a container pocket having an open end sized to receive the planar heat transfer pouch, the container made of a flexible material to conform the container against a surface of the body part;
an elongated strap made of a flexible material having a first end and a second end, wherein the first end of the strap is affixed to the container; and
a fastener attached to the strap at the second strap end and engageable with the container material to secure the container against the surface of the body part when the strap is wrapped around the body part.

12. A device as recited in claim 11 further comprising a thermal barrier positioned in the pocket to interpose the heat transfer pouch between the thermal barrier and the body part.

13. A device as recited in claim 11 wherein the substantially planar heat transfer pouch comprises a segmented pouch having a periphery, the segmented pouch being formed with a plurality of substantially coplanar pouch pockets for holding a respective plurality of frozen fluid lumps, wherein the pouch is flexible to conform the pouch against a surface of the body part.

14. A device as recited in claim 11 wherein the pouch comprises a first layer bonded to a second layer to establish the pouch pockets, each layer made of a combination of nylon and polyethylene and being sufficiently stretchable to accommodate a freezing of the fluid into lumps within the respective pouch pockets.

15. A device as recited in claim 14 wherein the frozen fluid is ice.

16. A device as recited in claim 14 wherein the frozen fluid lumps are each defined by coplanar sides, and wherein each side of each frozen fluid lump is less than approximately one inch.

17. A method for manufacturing a reusable wrap for holding and securing a substantially planar heat transfer pouch to a body part, the method comprising the steps of:
heat bonding an edge of a lower layer with an edge of an upper layer to establish a container formed as a pocket having an open end sized to receive the planar heat transfer pouch;
attaching an elongated strap made of a flexible material having a first end and a second end, wherein the first end of the strap is affixed to the container adjacent the container pocket open end;
positioning a thermal barrier in the pocket to interpose the heat transfer pouch between the thermal barrier and the body part; and
attaching a fastener to the strap at the second strap end, the fastener engageable with the container material to secure the container against the surface of the body part when the strap is wrapped around the body part.

18. A method as recited in claim 17 wherein the fastener is engageable with the strap material to secure the container against the surface of the body part when the strap is wrapped around the container.

19. A method as recited in claim 17 wherein the container comprises:
an upper layer having a periphery;
a lower layer having a periphery dimensioned and sectioned substantially the same as the upper layer, wherein a portion of the respective peripheral sections are permanently joined together to establish the pocket.

20. A method as recited in claim 17 wherein the container is stretchable and is made of a nonwoven material.
